(19) United States
(12) Patent Application Publication
(10) Pub. No.: US 2019/0113492 A1
(43) Pub. Date: Apr. 18, 2019

(54) INTEGRATION OF CALIBRATION CERTIFICATE IN GAS DETECTION DEVICES

(71) Applicant: Honeywell International Inc., Morris Plains, NJ (US)

(72) Inventor: Jesper Jensen, Dubai (AE)

(21) Appl. No.: 16/091,902

(22) PCT Filed: Apr. 7, 2016
(86) PCT No.: PCT/US2016/026321

§ 371 Date: Oct. 5, 2018

(51) Int. Cl. G01N 33/00

(52) U.S. Cl. CPC G01N 33/0008

(57) ABSTRACT

Embodiments relate generally to methods and systems for communicating, storing, and accessing a calibration and test certificate for a gas detector. A calibration and test certificate may verify that the gas detector has been tested and/or calibrated and can be operated in compliance with specific regulations. Embodiments of the disclosure include integrating the calibration certificate into the individual device, where it may be readily viewed by the user, a supervisor, or inspector to verify the compliance of the device. The certificate may be required to be verified when a user takes the gas detector to a worksite before work can begin. Therefore, storing the certificate on the device may simplify the process of accessing the certificate for the user and supervisor.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Gas detectors may be carried by workers in a variety of work environments. Gas detectors may be used to alert the user to the presence of harmful gases in the environment. To ensure that the sensors in gas detectors are working properly, the gas detectors may be periodically tested, calibrated, and/or inspected.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 illustrates a communication system according to an embodiment of the disclosure;

FIG. 2 illustrates a gas detector comprising a display according to an embodiment of the disclosure;

FIGS. 3A-3B illustrate illustrates a gas detector comprising a display according to an embodiment of the disclosure; and

FIG. 4 illustrates a calibration and test certificate according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example;

The terms “about” or “approximately” or the like, when used with a number, may mean that specific number, or alternatively, a range in proximity to the specific number, as understood by persons of skill in the art field; and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Embodiments of the disclosure include methods and systems for communicating, storing, and accessing a calibration and test certificate for a gas detector. Measuring devices comprising sensors, such as gas detectors, require calibration and testing to ensure that the sensors are working correctly. A calibration and test certificate may be generated, or issued, when calibration or testing is completed on the gas detector. The calibration and test certificate may verify that the gas detector can be operated in compliance with specific regulations. Typically, these certificates may be stored remotely, either in paper form or electronically. Also, the device may comprise some kind of indication for the user that the device is, or is not, in compliance, which may be a light or display indicator of some kind. In some cases, a portion of the calibration data may be stored locally on the device, but this may not include the verified and official calibration certificate.

Embodiments of the disclosure include integrating the calibration certificate into the individual device, where it may be readily viewed by the user, a supervisor, or inspector to verify the compliance of the device. The certificate may be required to be verified when a user takes the gas detector to a worksite before work can begin. Therefore, storing the certificate on the device may simplify the process of accessing the certificate for the user and supervisor.

Additionally, if the gas detector is a rental unit, it may be used by a variety of users between calibrations. Storing the calibration certificate locally on the device may simplify the process of accessing the certificate for a new user. For example, gas detectors carried by workers on a drilling rig may be inspected by the company running the rig. If the devices do not have the required certificates, work cannot begin. However, the devices may be tested and calibrated at another location, requiring remote access and/or transfer of the calibration certificates for each of the specific devices in use. However, if the calibration certificate is stored by the gas detector, it may be accessed at any time, either locally on the gas detector, or by wireless communication with a handheld device.

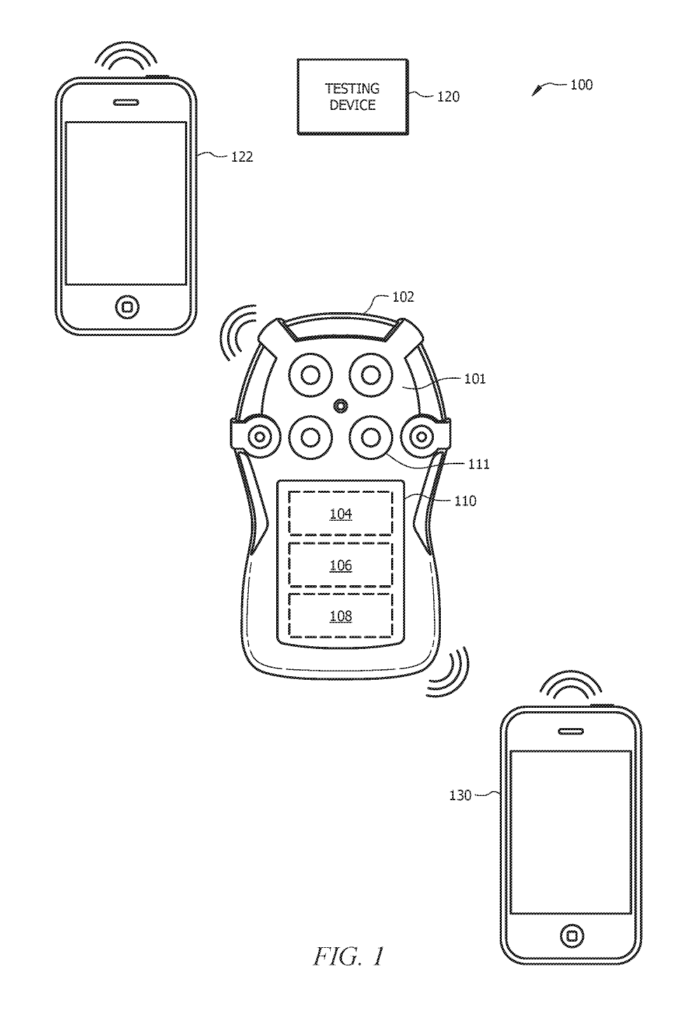

Referring now to FIG. 1, an exemplary communication system 100 is shown. The system 100 may comprise a gas detector 102 operable to detect harmful gases in the environment, and alert a user when harmful gases are detected. In some embodiments, the gas detector 102 may comprise a memory 104, a processor 106 operable to access and store to the memory 104, and a wireless communication module 108. In some embodiments, the wireless module 108 may operable to send and receive wireless communication with other wireless devices. In some embodiments, the gas detector 102 may comprise a user interface 101, which may comprise buttons 111 and/or displays 110.

In some embodiments, the gas detector 102 may be calibrated and/or tested periodically to ensure that the sensors and other elements on the gas detector 102 are functioning properly. In some embodiments, the gas detector 102 may interact with a testing device 120 to complete a test or calibration on the device 102. In some embodiments, the testing may be completed by a user carrying a mobile device 122, where the mobile device 122 may also communicate with the gas detector 102.

In some embodiments, when testing is completed for the gas detector 102, the testing device 120 may generate a calibration and test certificate, verifying that the gas detector 102 is in compliance with the testing and calibration requirements. In some embodiments, the calibration and test certificate may contain a signature from the person who completed the testing and/or calibration on the gas detector 102. In some embodiments, the mobile device 122 may receive the calibration and test certificate from the testing device 120, and the mobile device 122 may forward the calibration and test certificate to the gas detector 102. In some embodiments, the processor 106 may receive the calibration and test certificate via the wireless module 108, and the processor 106 may store the calibration and test certificate in the memory 104 of the gas detector 102. In some embodiments, the processor 106 may receive the calibration and test certificate via a wired connection with the testing device 120 and/or the mobile device 122, and the processor 106 may store the calibration and test certificate in the memory 104 of the gas detector 102.

In some embodiments, when a user takes the gas detector 102 to be used in a work environment, the calibration and test certificate for the gas detector 102 may be verified before the gas detector 102 can be used. In some embodiments, the user may be able to access the calibration and test certificate from the memory 104 of the gas detector 102, and the certificate may be shown on a display 110 of the gas detector. In some embodiments, a mobile device 130 (which may be carried by the user and/or the supervisor) may receive the calibration and test certificate from the gas detector 102 via the wireless module 108 of the gas detector 102. Then, the certificate may be displayed by the mobile device 130. In some embodiments, the calibration and test certificate may be accessed another way, and somehow displayed to the user and/or supervisor, wherein the certificate may be access from the memory 104 of the gas detector 102.

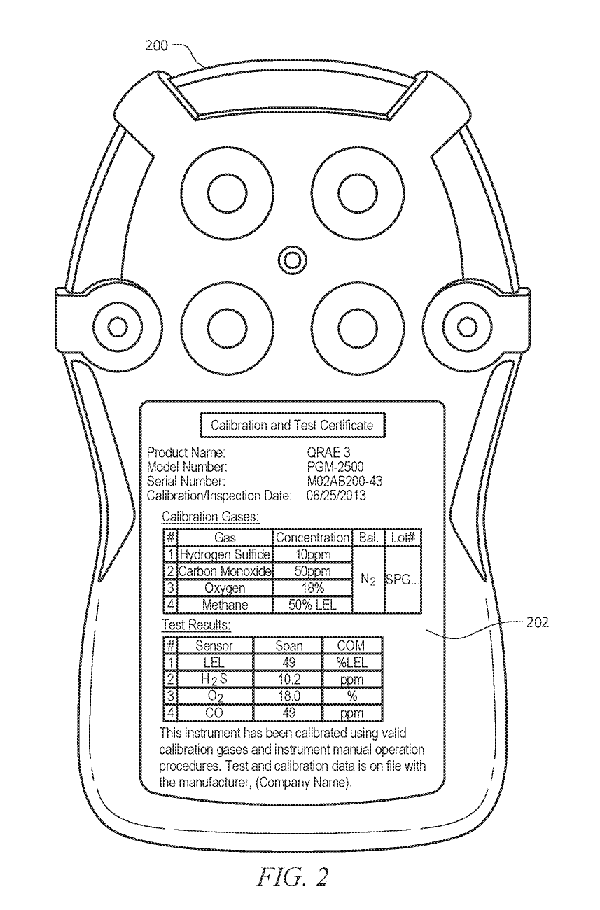

FIG. 2 illustrates an exemplary gas detector device 200 comprising a display 202. The display 202 may be a part of user interface of the gas detector device 200. In some embodiments, the display 202 may be operable to display the calibration certificate received by the device 200 during the calibration and testing of the device 200. A user may take the gas detector 200 to be used at a work site. In some embodiments, a supervisor at the work site may verify that the gas detector 200 is compliant with calibration and testing regulations, and therefore may wish to view the calibration and test certification. Instead of needing to remotely access the certificate, a user or supervisor may access the calibration certificate from the memory of the gas detector 200, wherein the calibration and test certificate may be shown on the display 202.

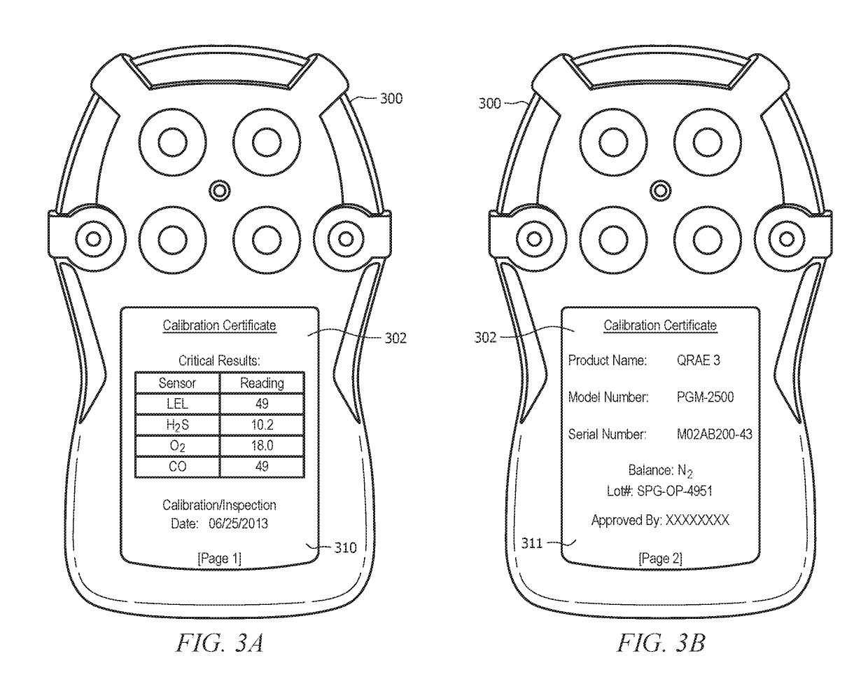

FIGS. 3A-3B illustrate another exemplary embodiment of a gas detector 300 comprising a display 302 operable to display a calibration and test certificate received by the gas detector 300 during the calibration and testing of the gas detector 300. A user may take the gas detector 300 to be used at a work site. In some embodiments, a supervisor at the work site may verify that the gas detector 300 is compliant with calibration and testing regulations, and therefore may wish to view the calibration and test certification. Instead of needing to remotely access the certificate, a user or supervisor may access the calibration certificate from the memory of the gas detector 300, wherein the calibration and test certificate may be shown on the display 302.

In some embodiments, the calibration certificate may contain too much information to be displayed as one screen. Therefore, the processor of the gas detector 300 may be operable to generate a plurality of display screens 310 and 311 containing the information from the calibration and test certificate. In other words, the calibration and test certificate may be displayed in parts, where the information may be split into multiple display screens 310 and 311. A user may switch between the screens 310 and 311 using the user interface of the gas detector 300. In some embodiments, the information that is considered most important may be included on a first screen 310, while other information may be included on a second screen 311.

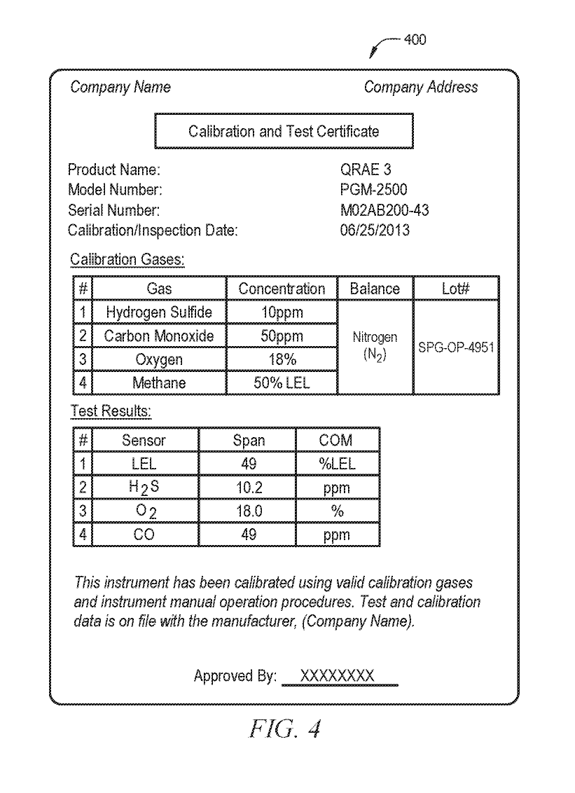

Referring now to FIG. 4, an exemplary calibration certificate 400 is shown. The calibration certificate may comprise testing and calibration information, for example, from the most recent calibration completed on a gas detector device. The calibration and test certificate may comprise the product name (or name of the gas detector), the model number of the gas detector, and the serial number of the gas detector. In some embodiments, this information may be received from the gas detector during the testing process. The calibration and test certificate may also comprise the date that calibration, testing, and/or inspection was completed.

In some embodiments, the calibration and test certificate may comprise information on the gases that are tested and/or calibrated. In some embodiments, this information may be shown in table form. In some embodiments, the calibration gas information may comprise the type of gas, the concentration of gas, the balance used, and the lot number of the gasses.

In some embodiments, the calibration and test certificate may comprise the test results, which may be shown in table form. In some embodiments, the test results may comprise the sensor name, the span of the test reading, and the units of measurement (UOM). In some embodiments, the calibration and test certificate may also comprise the name and/or signature of the person who completed the testing/calibration. In some embodiments, the calibration and test certificate may also comprise other identifying information, such as the name, address, and/or contact information for the company that performed the testing/calibration.

In some embodiments, calibration and test certificates may comprise more or less information than shown in FIG. 4. The certificate shown is meant to be an example of information that may be included in a calibration and test certificate that may be stored by a gas detector.

Embodiments of the disclosure may comprise a method for storing and communicating a calibration and test certificate comprising performing calibration or testing on a gas detector device; receiving, to the gas detector, a calibration and test certificate containing the data from the calibration or testing; storing, by the gas detector, the calibration and test certificate in the memory of the gas detector, wherein the calibration and test certificate contains a signature from the person who completed the testing; accessing the calibration and test certificate when requested by a user via a user interface of the gas detector; and displaying the calibration and test certificate on a display of the gas detector.

In some embodiments, a wireless module of the gas detector wirelessly receives the calibration and test certificate. In some embodiments, the gas detector wirelessly receives the calibration and test certificate from a mobile device carried by the user testing the device. In some embodiments, the method may further comprise sending the calibration and test certificate to a device carried by a supervisor, wherein the calibration and test certificate is displayed by the supervisor's device. In some embodiments, a wireless module of the gas detector wirelessly sends the calibration and test certificate to the device carried by the supervisor. In some embodiments, the method may further comprise assigning the gas detector device to a specific user. In some embodiments, the gas detector is temporarily assigned to a worker after the calibration and testing.

In some embodiments, the method may further comprise performing a second calibration or test on a gas detector device; receiving, to a gas detector, a second calibration and test certificate containing the data from the calibration or testing; updating, by the gas detector, the calibration and test certificate stored in the memory of the gas detector; accessing the second calibration and test certificate when requested by a user via a user interface of the gas detector; and displaying the second calibration and test certificate on a display of the gas detector. In some embodiments, the calibration and test certificate is displayed as a whole on the display of the gas detector. In some embodiments, the method may further comprising generating a plurality of display screens containing parts of the calibration and test certificate data; and displaying the plurality of display screens on the display of the gas detector, wherein a user may switch between the display screens.

Additional embodiments of the disclosure may comprise a gas detector comprising a display; a memory; a processor connected to the memory operable to receive a calibration and test certificate containing data from calibration or testing completed on the gas detector, wherein the calibration and test certificate contains a signature from the person who completed the testing; store the calibration and test certificate to the memory of the gas detector; access the calibration and test certificate when requested by a user via a user interface of the gas detector; and sending the calibration and test certificate to the display of the gas detector.

In some embodiments, the gas detector may further comprise a wireless module, wherein the processor is further operable to wirelessly receive the calibration and test certificate. In some embodiments, the processor wirelessly receives the calibration and test certificate from a testing device. In some embodiments, the processor wirelessly receives the calibration and test certificate from a mobile device carried by the tester. In some embodiments, the processor is further operable to wirelessly send the calibration and test certificate to a device carried by a supervisor.

In some embodiments, the calibration and test certificate is displayed as a whole on the display of the gas detector. In some embodiments, the processor is further operable to generate a plurality of display screens containing parts of the calibration and test certificate data.

Other embodiments of the disclosure may comprise a method for storing and communicating a calibration and test certificate comprising performing calibration or testing on a gas detector; wirelessly receiving, by a wireless module of the gas detector, a calibration and test certificate containing the data from the calibration or testing, wherein the calibration and test certificate contains a signature from the person who completed the testing; storing the calibration and test certificate in the memory of the gas detector; accessing the calibration and test certificate when requested by a user via a user interface of the gas detector; and displaying the calibration and test certificate on a display of the gas detector.

In some embodiments, the method may further comprise sending, by the wireless module of the gas detector, the calibration and test certificate to a device carried by a supervisor, wherein the calibration and test certificate is displayed by the supervisor's device. In some embodiments, the method may further comprise generating a plurality of display screens containing parts of the calibration and test certificate; and displaying the plurality of display screens on the display of the gas detector, wherein a user may switch between display screens.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

1-15. (canceled)

16. A method for storing and communicating a calibration and test certificate comprising:
performing calibration or testing on a gas detector device;
receiving, to the gas detector, a calibration and test certificate containing the data from the calibration or testing, wherein the calibration and test certificate contains a signature from the person who completed the testing;
storing, by the gas detector, the calibration and test certificate in the memory of the gas detector;
accessing the calibration and test certificate when requested by a user via a user interface of the gas detector; and
displaying the calibration and test certificate on a display of the gas detector.

17. The method of claim 16, wherein a wireless module of the gas detector wirelessly receives the calibration and test certificate.

18. The method of claim 17, wherein the gas detector wireless receives the calibration and test certificate from a mobile device carried by the user testing the device.

19. The method of claim 16, further comprising sending the calibration and test certificate to a device carried by a supervisor, wherein the calibration and test certificate is displayed by the supervisor's device.

20. The method of claim 19, wherein a wireless module of the gas detector wirelessly sends the calibration and test certificate to the device carried by the supervisor.

21. The method of claim 16, further comprising assigning the gas detector device to a specific user.

22. The method of claim 16, wherein the gas detector is temporarily assigned to a worker after the calibration and testing.

23. The method of claim 16, further comprising:
performing a second calibration or test on a gas detector device;
receiving, to a gas detector, a second calibration and test certificate containing the data from the calibration or testing;
updating, by the gas detector, the calibration and test certificate stored in the memory of the gas detector,
accessing the second calibration and test certificate when requested by a user via a user interface of the gas detector; and
displaying the second calibration and test certificate on a display of the gas detector.

24. The method of claim 16, wherein the calibration and test certificate is displayed as a whole on the display of the gas detector.

25. The method of claim 16, further comprising:
generating a plurality of display screens containing parts of the calibration and test certificate data; and
displaying the plurality of display screens on the display of the gas detector, wherein a user may switch between the display screens.

26. A gas detector comprising:
a display;
a memory;
a processor connected to the memory operable to:
receive a calibration and test certificate containing data from calibration or testing completed on the gas detector, wherein the calibration and test certificate contains a signature from the person who completed the testing;
store the calibration and test certificate to the memory of the gas detector;
access the calibration and test certificate when requested by a user via a user interface of the gas detector; and
sending the calibration and test certificate to the display of the gas detector.

27. The gas detector of claim 26, further comprising a wireless module, wherein the processor is further operable to wirelessly receive the calibration and test certificate.

28. The gas detector of claim 27, wherein the processor wirelessly receives the calibration and test certificate from a testing device.

29. The gas detector of claim 27, wherein the processor wirelessly receives the calibration and test certificate from a mobile device carried by the tester.

30. The gas detector of claim 27, wherein the processor is further operable to wirelessly send the calibration and test certificate to a device carried by a supervisor.

31. The gas detector of claim 26, wherein the calibration and test certificate is displayed as a whole on the display of the gas detector.

32. The gas detector of claim 26, wherein the processor is further operable to generate a plurality of display screens containing parts of the calibration and test certificate data.

33. A method for storing and communicating a calibration and test certificate comprising:
performing calibration or testing on a gas detector;
wirelessly receiving, by a wireless module of the gas detector, a calibration and test certificate containing the data from the calibration or testing, wherein the calibration and test certificate contains a signature from the person who completed the testing;
storing the calibration and test certificate in the memory of the gas detector;
accessing the calibration and test certificate when requested by a user via a user interface of the gas detector; and
displaying the calibration and test certificate on a display of the gas detector.

34. The method of claim 18, further comprising sending, by the wireless module of the gas detector, the calibration and test certificate to a device carried by a supervisor, wherein the calibration and test certificate is displayed by the supervisor's device.

35. The method of claim 18, further comprising:
generating a plurality of display screens containing parts of the calibration and test certificate; and
displaying the plurality of display screens on the display of the gas detector, wherein a user may switch between the display screens.
